(19) United States
(12) Patent Application Publication
(10) Pub. No.: US 2021/0215698 A1
(43) Pub. Date: Jul. 15, 2021

(54) Urinary DNA Detection For Urothelial Cancer

(71) Applicant: Institute For Cancer Research d/b/a The Research Institute of Fox Chase Cancer Center, Philadelphia, PA (US)

(72) Inventor: Philip Abbosh, Philadelphia, PA (US)

(21) Appl. No.: 17/055,722

(22) PCT Filed: May 15, 2019
(86) PCT No.: PCT/US2019/032380

§ 371 Date: Nov. 16, 2020

Related U.S. Application Data
(60) Provisional application No. 62/673,172, filed on May 18, 2018.

(51) Int. Cl. G01N 33/574; A61P 35/00; A61K 31/704; C07K 16/28; A61K 31/337; A61K 31/282

(52) U.S. Cl. CPC G01N 33/57407; A61P 35/00; A61K 31/704; A61K 31/282; A61K 31/337; C07K 16/2827; C07K 16/2818

(57) ABSTRACT

The present disclosure provides methods of treating a subject having urothelial cancer, and methods of monitoring the efficacy of neoadjuvant treatment in a subject having clinically localized urothelial cancer.

### FIELD

The present disclosure is directed, in part, to methods of treating a subject having urothelial cancer, and methods of monitoring the efficacy of neoadjuvant treatment in a subject having clinically localized urothelial cancer.

### BACKGROUND

Radical cystectomy (RC), surgical removal of the bladder, is among the most complicated (Finks et al., N. Engl. J. Med., 2011, 364, 2128-37) and expensive (Avritscher et al., Urology, 2006, 68, 549-53; Botteman et al., Pharmacoeconomics, 2003, 21, 1315-30; and Riley et al., Med. Care, 1995, 33, 828-41) cancer surgeries. RC is associated with a 30-40% major complication rate (Hautmann et al., Nat. Rev. Urol., 2011, 8, 667-77; and Novara et al., J. Urol., 2009, 182, 914-21), up to a 7% 90-day mortality (Cancer Facts and Figures, 2015; Gregg et al., J. Urol., 2011, 185, 90-6), and necessitates urinary diversion typically requiring a permanent ostomy, which is life-altering. In spite of the aggressive nature of muscle-invasive bladder cancer (MIBC), 30-40% of patients undergoing neoadjuvant chemotherapy (NAC) will have pathologic complete response (pCR) at the time of RC (Grossman et al., N. Engl. J. Med., 2003, 349, 859-66; Plimack et al., J. Clin. Oncol., 2014, 32, 1895-901; and Petrelli et al., Eur. Urol., 2014, 65, 350-7). NAC followed by RC is the current standard of care. These patients may be cured in absence of bladder removal, but currently, the only reliable way to determine if a patient has had a complete response after NAC is to remove the bladder and examine it pathologically. Prior attempts at organ sparing after current clinical assessment tools suggest that pCR was achieved have resulted in unacceptably high failure rates, as measured by any recurrence, muscle-invasive recurrence, metastasis, bladder cancer death, or salvage cystectomy (Herr, Eur. Urology, 2008, 54, 126-132; Meyer et al., J. Urology, 2014, 192, 1-6; and Herr et al., J. Clin. Oncol., 1998, 16, 1298-1301). This high failure rate dictates organ removal for most urologists/patients to avoid leaving a cancerous bladder in situ.

Patients can also develop urothelial cancers of the upper urinary tract (UTUC), which can carry an even worse long-term survival. Similarly though, when removal of the kidney and ureter (radical nephroureterectomy; RNU) is preceded by NAC, 10-20% of patients will have achieved a pCR. Although RNU is not as complicated as RC and does not require urinary diversion, removal of renal units has its own set of sequelae including kidney failure, worsening of other cardiovascular comorbidities, and requirement for hemodialysis in some cases. Complicating the manner is that the clinical grading and staging of UTUC is difficult due to the anatomy of the collecting system and the tiny instrumentation that is required to obtain tissue biopsy. Small pieces of tissue are obtained by the urologist and can often either be nondiagnostic or (worse) not representative of the entire tumor leading to understaging. These facts frame the most important questions to urologists performing RC and RNU: whether an individual patient's response to NAC can be predicted a priori or identified post hoc and, if so, whether RC or RNU can be safely avoided in such chemoresponders. Reliable answers to these two questions would facilitate safe radical surgery avoidance in MIBC and UTUC patients.

Organ preservation has become the standard-of-care in breast cancer (Litiere et al., Lancet Oncol., 2012, 13, 412-9; and Julien et al., Lancet, 2000, 355, 528-33), laryngeal cancer (The Department of Veterans Affairs Laryngeal Cancer Study Group, N. Engl. J. Med., 1991, 324, 1685-90), anal cancer (Nigro et al., Cancer, 1983, 51, 1826-9), and extremity sarcomas (Rosenberg et al., Ann. Surg., 1982, 196, 305-15), sparing significant morbidity without change in overall survival. But without a response biomarker, there is an obligatory association with higher local recurrence rate since some patients will not be true complete responders. Clinicopathological factors and radiological studies do not accurately identify MIBC or UTUC patients who achieve pCR; historically, the “biomarker of response” is the RC specimen. In patients deemed complete responders by bladder biopsy after NAC who avoid RC, there is a 30-50% rate of local failure necessitating salvage RC, and there is a similarly high rate of local failure after bladder preservation with chemoradiotherapy (James et al., N. Engl, J. Med., 2012, 366, 1477-88; and Mitin et al., Lancet Oncol., 2013, 14, 863-72; Mak et al., J. Clin. Oncol., 2014, 32, 3801-9). However, there is also a 50-70% rate of metastasis-free survival with intact bladder after RC avoidance (Herr, Eur. Urol., 2008, 54, 126-32; Meyer et al., J. Urol., 2014, 192, 696-701; Solsona et al., Eur. Urol., 2009, 55, 911-9; and Herr, J. Clin. Oncol., 2001, 19, 89-93).

Therefore, although there is a significant fraction of patients who failed to safely avoid RC, there is also a sizeable proportion who did safely avoid RC. Data surrounding organ preservation in UTUC is lacking. A better way to distill true responders from presumptive responders is clearly needed by the urology community in order to safely avoid surgery in chemoresponders. A biomarker, or a panel of biomarkers, that permits identification of true complete responders so that can potentially safely avoid organ removal would be a practice-changing breakthrough, completely avoiding perioperative morbidity and urinary diversion in a significant proportion of MIBC patients or sparing nephron removal of UTUC patients while possibly realizing a cost benefit. In addition, UTUC biomarkers could be used for both dynamic response measurement and to enhance clinical staging and grading of disease.

Persistent circulating mutations have been detected in colorectal cancer (Tie et al., Sci. Transl. Med., 2016, 8, 346ra92) and leukemia (Klco et al., J. Amer. Med. Assoc., 2015, 314, 811-22) patients after curative-intent therapy, and their presence was superior to any other clinicopathologic factors in predicting recurrence. The central premise of such studies is that patients with no residual disease will have no source of somatic mutations to shed into body fluids. Similarly, patients with superficial bladder cancer (as opposed to MIBC) demonstrating mutation clearance from urine or serum have longer cancer-free survival or are cured (Birkenkamp-Demtroder et al., Eur. Urol., 2016, 70, 75-82).

### SUMMARY

It is disclosed herein that mutation clearance from urinary DNA (uDNA) of patients with urothelial cancers who received NAC or other neoadjuvant therapies would associate with pCR, while those with mutation persistence would have residual disease.

The present disclosure provides methods of treating a subject having urothelial cancer comprising: analyzing a urine sample obtained from the subject for the presence of one or more mutations in one or more urothelial cancer-associated genes; wherein: i) if no mutations in urothelial cancer-associated genes are detected in the sample of urine, then refraining from removing the urinary bladder, ureter, or kidney, or any combination thereof, from the subject; and ii) if mutations in one or more urothelial cancer-associated genes are detected in the sample of urine, then further removing the urinary bladder, ureter, or kidney, or any combination thereof, from the subject.

The present disclosure also provides methods of treating a subject having urothelial cancer comprising: administering any neoadjuvant therapy to the subject; and analyzing a urine sample obtained from the subject after receiving neoadjuvant treatment for the presence of one or more mutations in one or more urothelial cancer-associated genes; wherein: i) if no mutations in urothelial cancer-associated genes are detected in the sample of urine, then refraining from removing the urinary bladder, ureter, or kidney, or any combination thereof, from the subject; and ii) if mutations in one or more urothelial cancer-associated genes are detected in the sample of urine, then further removing the urinary bladder, ureter, or kidney, or any combination thereof, from the subject.

The present disclosure also provides methods of monitoring the efficacy of neoadjuvant treatment in a subject having clinically localized urothelial cancer comprising: analyzing a urine sample obtained from the subject prior to neoadjuvant treatment for the presence of one or more mutations in one or more urothelial cancer-associated genes; administering neoadjuvant therapy to the subject; analyzing a urine sample obtained from the subject after neoadjuvant treatment for the presence of one or more mutations in one or more urothelial cancer-associated genes; and comparing the subject's somatic mutations detected in the urine samples obtained prior to and after administration of neoadjuvant therapy; wherein: i) if all the subject's somatic mutations in the one or more urothelial cancer-associated genes detected in the urine sample obtained prior to administering neoadjuvant therapy are not detected in the urine sample obtained after administering neoadjuvant therapy, then the neoadjuvant treatment is deemed successful, and the subject is not further suggested to have removal of the urinary bladder, ureter, or kidney, or any combination thereof, from the subject; and ii) if any of the subject's somatic mutations in the one or more urothelial cancer-associated genes detected in the urine sample obtained prior to administering neoadjuvant therapy are persistently detected in the urine sample obtained after administering neoadjuvant therapy, then the neoadjuvant treatment is deemed insufficient, and the subject is further recommended to have removal of the urinary bladder, ureter, or kidney, or any combination thereof, from the subject.

The present disclosure also provides methods of analyzing a subject having urothelial cancer comprising: analyzing a urine sample obtained from the subject prior to neoadjuvant treatment for the presence of one or more mutations in one or more urothelial cancer-associated genes; administering neoadjuvant therapy to the subject; analyzing a urine sample obtained from the subject after neoadjuvant treatment for the presence of one or more mutations in one or more urothelial cancer-associated genes; and comparing the subject's urine mutation profile detected in the urine samples obtained prior to and after administration of neoadjuvant therapy.

The present disclosure also provides methods of treating a subject having urothelial cancer comprising: requesting a test for the subject, wherein the test analyzes urine samples obtained from the subject prior to and after neoadjuvant treatment for one or more mutations in one or more urothelial cancer-associated genes; examining the result of the test, wherein the result indicates whether or not all of the mutations in the urothelial cancer-associated genes identified in the urine sample obtained prior to neoadjuvant treatment are absent from the urine sample obtained after neoadjuvant treatment; and i) if all the mutations in the urothelial cancer-associated genes identified in the urine sample obtained prior to neoadjuvant treatment are absent from the urine sample obtained after neoadjuvant treatment, then refraining from removing the urinary bladder, ureter, or kidney, or any combination thereof, from the subject; or ii) if all the mutations in the urothelial cancer-associated genes identified in the urine sample obtained prior to neoadjuvant treatment are not absent from the urine sample obtained after neoadjuvant treatment, then further removing the urinary bladder, ureter, or kidney, or any combination thereof, from the subject.

The present disclosure also provides methods of treating a subject having urothelial cancer comprising: examining the result of a test for the subject, wherein the test analyzes urine samples obtained from the subject prior to and after neoadjuvant treatment for one or more mutations in one or more urothelial cancer-associated genes, and wherein the result indicates whether or not all of the mutations in the urothelial cancer-associated genes identified in the urine sample obtained prior to neoadjuvant treatment are absent from the urine sample obtained after neoadjuvant treatment; and i) if all the mutations in the urothelial cancer-associated genes identified in the urine sample obtained prior to neoadjuvant treatment are absent from the urine sample obtained after neoadjuvant treatment, then refraining from removing the urinary bladder, ureter, or kidney, or any combination thereof, from the subject; or ii) if all the mutations in the urothelial cancer-associated genes identified in the urine sample obtained prior to neoadjuvant treatment are not absent from the urine sample obtained after neoadjuvant treatment, then further removing the urinary bladder, ureter, or kidney, or any combination thereof, from the subject.

### BRIEF DESCRIPTION OF THE DRAWINGS

FIG. 1 shows representative genes that are present in the sequencing panel and representative rationale for inclusion of the gene (i.e. nominated as a bladder cancer driver from TCGA I (Weinstein et al, Nature, 2014, 507, 315-22), TCGA II (Robertson et al, Cell, 2017, 170, 1-17), or commonly mutated in other cancer and in bladder cancer (Lawrence et al, Nature, 2014, 505, 495-501)).

FIG. 2 shows results of analysis of benchmarked MIBC patients (Upper Panel) and unbenchmarked MIBC patients (Lower Panel); study of benchmarked patients' urine samples demonstrates that tissue mutations (M_{T}) are detectable in uDNA; for benchmark MIBC cases, the denominator in each cell in the table refers to the number of mutations identified in whole exome sequencing from prechemotherapy bladder cancer tissue (i.e. M_{T}); the numerator in each cell refers to the number of M_{T} that were detectable in each urine specimen (i.e. M_{U}) at the specified time point; red cells indicate that somatic mutations in the gene panel were detectable in urine at that time point, while green cells indicate that somatic mutations in the gene panel were absent; ‘(+1)’ refers to presence of TERT promoter hotspot (Horn et al, Science, 2013, 339, 959-961) which was not detected by whole exome sequencing due to its position in the noncoding region; all patients received neoadjuvant dose-dense methotrexate/vinblastine/doxorubicin/cisplatin.

FIG. 3 shows a correlation of mutation clearance with responder status in UTUC cases which did not have benchmark whole exome sequencing; ‘Pre’ column documents the number of mutations which were detected in uDNA prior to neoadjuvant dose-dense methotrexate/vinblastine/doxorubicin/cisplatin; ‘Post’ column refers to the number of pre-chemotherapy mutations that were present after chemotherapy; red cells indicate that somatic mutations in the gene panel were detectable in urine at that time point, while green cells indicate that somatic mutations in the gene panel were absent.

FIG. 4 shows clinical and pathologic staging, treatment information, and other relevant information for patients from an independent cohort from another institution; all patients had analysis of a single pre-surgical urine specimen; molecular status indicates how many mutations were identified in the pre-extirpation urine sample; red cells indicate that somatic mutations in the gene panel were detectable in urine at that time point, while green cells indicate that somatic mutations in the gene panel were absent; GC=neoadjuvant gemcitabine/cisplatin; GC+pembro=neoadjuvant gemcitabine/cisplatin plus pembrolizumab; RC=radical cystectomy; RNU=radical nephroureterectomy; ddMVAC=dose dense methotrexate/vinblastine/doxorubicin/cisplpatin; ‘**’ designates that patient CL-16 had mutation in CDKN1A, which is associated with high-grade disease.

FIG. 5 describes the performance of the urinary test in identifying residual disease in a pathological specimen using a single urine sample taken prior to the time of radical cystectomy or radical nephroureterectomy.

### DESCRIPTION OF EMBODIMENTS

The terminology used herein is for the purpose of describing particular embodiments only and is not intended to be limiting.

Unless defined otherwise, all technical and scientific terms have the same meaning as is commonly understood by one of ordinary skill in the art to which the disclosed embodiments belong.

As used herein, the terms “a” or “an” mean “at least one” or “one or more” unless the context clearly indicates otherwise.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the terms “individual,” “subject,” and “patient,” used interchangeably, mean any animal described herein.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable sub-combination.

The present disclosure provides methods of treating a subject having urothelial cancer comprising: analyzing a urine sample obtained from the subject for the presence of one or more mutations in one or more urothelial cancer-associated genes; wherein: i) if no mutations in urothelial cancer-associated genes are detected in the sample of urine, then refraining from removing the urinary bladder, ureter, or kidney, or any combination thereof, from the subject; and ii) if mutations in one or more urothelial cancer-associated genes are detected in the sample of urine, then further removing the urinary bladder, ureter, or kidney, or any combination thereof, from the subject. In some embodiments, the analysis of the urine sample obtained from the subject occurs prior to chemotherapy. In some embodiments, the analysis of the urine sample obtained from the subject occurs between chemotherapy and an anticipated surgery date.

The present disclosure also provides methods of treating a subject having urothelial cancer comprising: a) administering neoadjuvant therapy to the subject; and b) analyzing a urine sample obtained from the subject after receiving neoadjuvant treatment for the presence of one or more mutations in one or more urothelial cancer-associated genes; wherein: i) if no mutations in urothelial cancer-associated genes are detected in the sample of urine, then refraining from removing the urinary bladder, ureter, or kidney, or any combination thereof, from the subject; and ii) if mutations in one or more urothelial cancer-associated genes are detected in the sample of urine, then further removing the urinary bladder, ureter, or kidney, or any combination thereof, from the subject.

In some embodiments, the methods further comprise analyzing a urine sample from the subject obtained prior to administering neoadjuvant therapy to the subject for the presence of one or more mutations in one or more urothelial cancer-associated genes. In some embodiments, the methods further comprise comparing the subject's urine mutation profile detected in the urine samples obtained prior to and after administration of neoadjuvant therapy. If all of the mutations in one or more urothelial cancer-associated genes detected in the urine sample obtained prior to administering neoadjuvant therapy are not also detected in the urine sample obtained after administering neoadjuvant therapy, then removal of the urinary bladder, ureter, or kidney, or any combination thereof, from the subject is not undertaken. If, however, any of the mutations in one or more urothelial cancer-associated genes detected in the urine sample obtained prior to administering neoadjuvant therapy are also detected in the urine sample obtained after administering neoadjuvant therapy, then removal of the urinary bladder, ureter, or kidney, or any combination thereof, from the subject is undertaken.

In some embodiments, the urothelial cancer is a muscle-invasive invasive urothelial cancer of the bladder, a non-muscle invasive urothelial cancer of the bladder, a ureteral cancer, or a renal pelvis cancer, or any combination thereof. In some embodiments, the urothelial cancer is a muscle-invasive invasive urothelial cancer of the bladder. In some embodiments, the urothelial cancer is a non-muscle invasive urothelial cancer of the bladder. In some embodiments, the urothelial cancer is a ureteral cancer. In some embodiments, the urothelial cancer is a renal pelvis cancer. In some embodiments, the urothelial cancer is urethral cancer.

In some embodiments, the urothelial cancer is urothelial carcinoma, squamous cell carcinoma, small cell carcinoma, adenocarcinoma, micropapillary carcinoma, plasmacytoid carcinoma, or sarcomatoid differentiation, or any combination thereof. In some embodiments, the urothelial cancer is urothelial carcinoma. In some embodiments, the urothelial cancer is squamous cell carcinoma. In some embodiments, the urothelial cancer is small cell carcinoma. In some embodiments, the urothelial cancer is adenocarcinoma. In some embodiments, the urothelial cancer is micropapillary carcinoma. In some embodiments, the urothelial cancer is plasmacytoid carcinoma. In some embodiments, the urothelial cancer is sarcomatoid differentiation.

In some embodiments, the urine sample is whole urine. In some embodiments, the urine sample is urine sediment or urine supernatant. In some embodiments, where the urine sample is obtained prior to neoadjuvant treatment, the urine sample is obtained from the subject two weeks, one week, three days, two days, or one day before the subject receives the first round of neoadjuvant treatment. In some embodiments, where the urine sample is obtained after neoadjuvant treatment, the urine sample is obtained from the subject four weeks, three weeks, two weeks, one week, three days, two days, or one day after the subject receives the first round of neoadjuvant treatment, or after the subject receives any round of neoadjuvant treatment, or after the subject receives the last round of neoadjuvant treatment.

As used herein, the phrase “neoadjuvant treatment” is meant to include all forms of treatment of cancer prior to a potential extirpative procedure including, but not limited to, traditional chemotherapy (i.e., anti-cancer agents or chemotherapeutic agents, whether they are administered parenterally or orally), immunotherapy (i.e., adoptive immunotherapy, CAR-T cell therapy, immune checkpoint blockade with small molecules or antibodies), small molecule enzyme or kinase inhibitors, intravesical therapies, antibody inhibitors of receptors or kinases, antibody-drug conjugates, and radiation therapy. In some embodiments, the chemotherapeutic agent is a platinum-based chemotherapeutic agent such as, for example, cisplatin, carboplatin, oxaliplatin, nedaplatin, triplatin tetranitrate, phenanthriplatin, picoplatin, or satraplatin, or any combination thereof. In some embodiments, the chemotherapeutic agent is methotrexate, vincristine, vinblastine, doxorubicin, tunicamycin, oligomycin, bortezomib, MG132, 5-flurouracil, sorafenib, or flavopiridol, gemcitabine, or any combination thereof. In some embodiments, the chemotherapeutic treatment is a combination of agents, such as, for example, methotrexate/vinblastine/doxorubicin/cisplatin (MVAC) or gemcitabine/cisplatin. In some embodiments, the chemotherapeutic treatment is an immunotherapeutic agent such as, for example, OPDIVO® (nivolumab), KEYTRUDA® (pembrolizumab), TECENTRIQ® (atezolizumab), IMFINZI® (durvalab), YERVOY® (ipilumumab), or BAVENCIO® (avelumab), or intravesical therapies such as Bacillus-Calmette Guerin, doxorubicin, cisplatin, gemcitabine, or docetaxel, thiotepa, valrubicin or any combination thereof. In some embodiments, the chemotherapeutic treatment is an immunotherapeutic agent such as, for example, nivolumab, pembrolizumab, atezolizumab, durvalab, ipilumumab, or avelumab.

In some embodiments, urinary DNA (uDNA) in the urine sample is analyzed for the presence of one or more mutations in one or more urothelial cancer-associated genes. The uDNA in the urine sample may have one, two, three, four, five, six, seven, eight, nine, ten, or more than ten mutations in one or more urothelial cancer-associated genes. The uDNA in the urine sample may have such mutations in one, two, three, four, five, six, seven, eight, nine, ten, or more than ten urothelial cancer-associated genes.

In some embodiments, at least 5 urothelial cancer-associated genes are analyzed for the presence of one or more somatic mutations. In some embodiments, at least 10 urothelial cancer-associated genes are analyzed for the presence of one or more somatic mutations. In some embodiments, at least 15 urothelial cancer-associated genes are analyzed for the presence of one or more somatic mutations. In some embodiments, at least 20 urothelial cancer-associated genes are analyzed for the presence of one or more somatic mutations. In some embodiments, at least 25 urothelial cancer-associated genes are analyzed for the presence of one or more somatic mutations. In some embodiments, at least 30 urothelial cancer-associated genes are analyzed for the presence of one or more somatic mutations. In some embodiments, at least 35 urothelial cancer-associated genes are analyzed for the presence of one or more somatic mutations. In some embodiments, at least 40 urothelial cancer-associated genes are analyzed for the presence of one or more somatic mutations. In some embodiments, at least 45 urothelial cancer-associated genes are analyzed for the presence of one or more somatic mutations. In some embodiments, at least 50 urothelial cancer-associated genes are analyzed for the presence of one or more somatic mutations. In some embodiments, at least 55 urothelial cancer-associated genes are analyzed for the presence of one or more somatic mutations.

In some embodiments, the urothelial cancer-associated mutations are in TCGA-nominated urothelial cancer driver genes, TERT promoter, or genes identified as significantly mutated across all cancers which are also common in urothelial cancers. In some embodiments, the urothelial cancer-associated mutations are in TCGA-nominated urothelial cancer driver genes. In some embodiments, the urothelial cancer-associated mutations are in the TERT promoter. In some embodiments, the urothelial cancer-associated mutations are in genes identified as significantly mutated across all cancers which are also common in urothelial cancers.

In some embodiments, the TCGA-nominated urothelial cancer driver genes are selected from the group consisting of ARID1A, ASXL1, ASXL2, ATM, BTG2, CASP8, CCND3, CDKN1A, CDKN2A, CREBBP, CTNNB1, ELF3, EP300, ERBB2, ERBB3, ERCC2, FAT1, FBXW7, FGFR3, FOXA1, FOXQ1, HRAS, KDM6A, KLF5, KMT2A, KMT2C, KMT2D, KRAS, NFE2L2, NRAS, PIK3CA, PTEN, RB1, RHOA, RHOB, RXRA, SPTAN, STAG2, TP53, TSC1, TXNIP, and ZFP36L1. In some embodiments, the TCGA-nominated urothelial cancer driver genes are selected from the group consisting of TP53, KDM6A, KMT2D, ARID1A, KMT2C, RB1, EP300, FGFR3, STAG2, and ATM.

In some embodiments, the mutation is in the upstream promoter of the TERT gene. TERT promoter mutations predominantly affect two hot spots, g.Chr5:1295228 C>T and g.Chr5:1295250 C>T (referring to the genomic sequence in GRCh38/hg38), which generate CCGGAA/T or GGAA/T motifs.

In some embodiments, the genes identified as significantly mutated across all cancers which are also common in urothelial cancers are selected from the group consisting of BAP1, BRCA1, BRCA2, ERCC4, FANCA, FANCC, FANCD2, KMT2B, NF1, PAIP1, PBRM1, RAD51, and SETD2.

In any of the one or more urothelial cancer-associated genes described herein, the specific mutation can be any mutation in the indicated gene. In some embodiments, the specific mutation can occur anywhere throughout the entire coding region, resulting in a loss of function, a partial loss of function, no loss in function, or an unknown effect on function (variant of uncertain significance). In some embodiments, the mutation is a missense mutation, a frameshifting mutation, a splice-site mutation, a nonsense mutation, a complex mutation, or a silent (synonymous) mutation. The specific mutation can be a driver mutation (i.e., causative of the cancer) or can be a passenger mutation (i.e., although not causative of the cancer, it is a biomarker for the cancer). Specific mutations in urothelial cancer-associated genes are known to those skilled in the art.

The detection of the one or more mutations in the one or more urothelial cancer-associated genes can be carried out by conventional means known in the art. In some embodiments, the presence of the one or more mutations in the one or more urothelial cancer-associated genes is detected by procedures such as, for example, nucleic acid sequencing, in situ hybridization, and immunohistochemistry, any of which may also involve nucleic acid amplification. Representative examples of nucleic acid sequencing techniques include, but are not limited to, chain terminator (Sanger) sequencing, dye terminator sequencing, sequencing by synthesis, pyrosequencing, and next-generation sequencing. Procedures for nucleic acid hybridization include using labeled primers or probes directed against one or more urothelial cancer-associated genes, and fixed cell preparations (fluorescence in situ hybridization). In some methods, a target urothelial cancer-associated gene may be amplified prior to or simultaneous with detection. Representative examples of nucleic acid amplification procedures include, but are not limited to, polymerase chain reaction (PCR), ligase chain reaction (LCR), strand displacement amplification (SDA), and nucleic acid sequence based amplification (NASBA). Procedures for detecting mutations in one or more urothelial cancer-associated genes include, for example, Southern blot hybridization, in situ hybridization, and fluorescence in situ hybridization (FISH).

In any of the embodiments described herein, the detection of one or more mutations in the one or more urothelial cancer-associated genes can be carried out by any of these procedures on a urine sample obtained from the subject. Performance of any of these procedures on a urine sample obtained from the subject can be compared to samples or sequence information from wild type (i.e., non-cancerous or germline) urothelial cancer-associated genes.

In some embodiments, the methods further comprise comparing the one or more mutations in the urothelial cancer-associated genes in the urine sample to germline mutations to filter out single nucleotide polymorphisms (SNPs), sequencing errors, and/or rare variants.

In some embodiments, the methods further comprise treating the subject with an organ preservation regimen if no mutations in urothelial cancer-associated genes are detected in the sample of urine obtained after administration of neoadjuvant therapy. In some embodiments, the organ preservation regimen comprises avoidance of radical cystectomy with or without urinary diversion, avoidance of radical nephroureterectomy, avoidance of distal ureterectomy and neocystostomy, or avoidance of irradiation of the bladder, renal pelvis, and/or ureter.

The present disclosure also provides methods of monitoring the efficacy of neoadjuvant therapeutic treatment in a subject having clinically localized urothelial cancer comprising: a) analyzing a urine sample obtained from the subject prior to neoadjuvant therapeutic treatment for the presence of one or more mutations in one or more urothelial cancer-associated genes; b) administering neoadjuvant therapy to the subject; c) analyzing a urine sample obtained from the subject after neoadjuvant treatment for the presence of one or more mutations in one or more urothelial cancer-associated genes; and d) comparing the subject's somatic mutations detected in the urine samples obtained prior to and after administration of neoadjuvant therapy; wherein: i) if all the subject's somatic mutations in the one or more urothelial cancer-associated genes detected in the urine sample obtained prior to administering neoadjuvant therapy are not detected in the urine sample obtained after administering neoadjuvant therapy, then the neoadjuvant therapeutic treatment is deemed successful, and the subject is not further recommended to undergo removal of the urinary bladder, ureter, or kidney, or any combination thereof, from the subject; and ii) if any of the subject's somatic mutations in the one or more urothelial cancer-associated genes detected in the urine sample obtained prior to administering neoadjuvant therapy are also detected in the urine sample obtained after administering neoadjuvant therapy, then the neoadjuvant therapeutic treatment is deemed insufficient, and the subject is further recommended to undergo removal of the urinary bladder, ureter, or kidney, or any combination thereof, from the subject.

In some embodiments, when none of the subject's somatic mutations are detected in the one or more urothelial cancer-associated genes, and/or the neoadjuvant therapeutic treatment is deemed successful, and the subject is not further recommended to undergo removal of the urinary bladder, ureter, or kidney, or any combination thereof, from the subject, the subject is further monitored by the methods described herein. In some embodiments, when all the subject's somatic mutations in the one or more urothelial cancer-associated genes detected in the urine sample obtained prior to administering neoadjuvant therapy are not detected in the urine sample obtained after administering neoadjuvant therapy, and the neoadjuvant therapeutic treatment is deemed successful, and the subject is not further recommended to undergo removal of the urinary bladder, ureter, or kidney, or any combination thereof, from the subject, the subject is further monitored by the methods described herein.

For example, when a patient is deemed eligible for exemption from organ removal, the patient typically undergoes a history and physical examination, and cystoscopy (endoscopic camera in the bladder) or ureteroscopy (endoscopic camera into the ureter/renal pelvis), or both, and urine cytology, with imaging at regular intervals after being spared organ removal. This is carried out because of the high failure rate of current clinical assessment tools available. If and when residual disease is identified after a period of attempted organ sparing, the patient undergoes radical organ(s) removal. The methods described herein using a urine sample may be more sensitive than current clinical assessment tools based on the current literature. The present methods may be used to identify patients for organ sparing approaches, and these patients will require surveillance to identify any recurrences which would preclude them from continued organ sparing.

In some embodiments, the urine analysis can be used as part of a surveillance strategy (in addition to H&P and cystoscopy/ureteroscopy, cytology, and/or imaging) for patients who retain their bladder after having a urine test showing that there is absence of somatic mutations in their urine test.

The urothelial cancer can be any of the urothelial cancers described herein, and the urine samples can be any of the urine samples described herein. The uDNA in the urine samples can be analyzed as described herein. The urothelial cancer-associated genes, and panels thereof, can be any of the urothelial cancer-associated genes, and panels thereof, described herein. The neoadjuvant treatment can be any of the neoadjuvant treatments described herein. The presence of the one or more mutations in the one or more urothelial cancer-associated genes can be detected by any of the procedures described herein. The methods may further comprise comparing the one or more mutations in the urothelial cancer-associated genes in the urine sample to germline mutations to filter out single nucleotide polymorphisms (SNPs), sequencing errors, and rare variants. The methods may further comprise treating the subject with any of the organ preservation regimens described herein if no mutations in urothelial cancer-associated genes are detected in the sample of urine obtained after administration of neoadjuvant therapy.

The present disclosure also provides methods of analyzing a subject having urothelial cancer comprising: a) analyzing a urine sample obtained from the subject prior to neoadjuvant treatment for the presence of one or more mutations in one or more urothelial cancer-associated genes; b) administering neoadjuvant therapy to the subject; c) analyzing a urine sample obtained from the subject after neoadjuvant treatment for the presence of one or more mutations in one or more urothelial cancer-associated genes; and d) comparing the subject's urine mutation profile detected in the urine samples obtained prior to and after administration of neoadjuvant therapy. The urothelial cancer can be any of the urothelial cancers described herein, and the urine samples can be any of the urine samples described herein. The uDNA in the urine samples can be analyzed as described herein. The urothelial cancer-associated genes, and panels thereof, can be any of the urothelial cancer-associated genes, and panels thereof, described herein. The neoadjuvant treatment can be any of the neoadjuvant treatments described herein. The presence of the one or more mutations in the one or more urothelial cancer-associated genes can be detected by any of the procedures described herein. The methods may further comprise comparing the one or more mutations in the urothelial cancer-associated genes in the urine sample to germline mutations to filter out single nucleotide polymorphisms (SNPs), sequencing errors, and rare variants.

The present disclosure also provides methods of treating a subject having urothelial cancer comprising: a) requesting a test for the subject, wherein the test analyzes urine samples obtained from the subject prior to and after neoadjuvant treatment for one or more mutations in one or more urothelial cancer-associated genes; b) examining the result of the test, wherein the result indicates whether or not all of the mutations in the urothelial cancer-associated genes identified in the urine sample obtained prior to neoadjuvant treatment are absent from the urine sample obtained after neoadjuvant treatment; and i) if all the mutations in the urothelial cancer-associated genes identified in the urine sample obtained prior to neoadjuvant treatment are absent from the urine sample obtained after neoadjuvant treatment, then refraining from removing the urinary bladder, ureter, or kidney, or any combination thereof, from the subject; or ii) if all the mutations in the urothelial cancer-associated genes identified in the urine sample obtained prior to neoadjuvant treatment are not absent from the urine sample obtained after neoadjuvant treatment, then further removing the urinary bladder, ureter, or kidney, or any combination thereof, from the subject. The urothelial cancer can be any of the urothelial cancers described herein, and the urine samples can be any of the urine samples described herein. The uDNA in the urine samples can be analyzed as described herein. The urothelial cancer-associated genes, and panels thereof, can be any of the urothelial cancer-associated genes, and panels thereof, described herein. The neoadjuvant treatment can be any of the neoadjuvant treatments described herein. The presence of the one or more mutations in the one or more urothelial cancer-associated genes can be detected by any of the procedures described herein. The methods may further comprise comparing the one or more mutations in the urothelial cancer-associated genes in the urine sample to germline mutations to filter out single nucleotide polymorphisms (SNPs), sequencing errors, and rare variants.

The present disclosure also provides methods of treating a subject having urothelial cancer comprising: a) examining the result of a test for the subject, wherein the test analyzes urine samples obtained from the subject prior to and after neoadjuvant treatment for one or more mutations in one or more urothelial cancer-associated genes, and wherein the result indicates whether or not all of the mutations in the urothelial cancer-associated genes identified in the urine sample obtained prior to neoadjuvant treatment are absent from the urine sample obtained after neoadjuvant treatment; and i) if all the mutations in the urothelial cancer-associated genes identified in the urine sample obtained prior to neoadjuvant treatment are absent from the urine sample obtained after neoadjuvant treatment, then refraining from removing the urinary bladder, ureter, or kidney, or any combination thereof, from the subject; or ii) if all the mutations in the urothelial cancer-associated genes identified in the urine sample obtained prior to neoadjuvant treatment are not absent from the urine sample obtained after neoadjuvant treatment, then further removing the urinary bladder, ureter, or kidney, or any combination thereof, from the subject. The urothelial cancer can be any of the urothelial cancers described herein, and the urine samples can be any of the urine samples described herein. The uDNA in the urine samples can be analyzed as described herein. The urothelial cancer-associated genes, and panels thereof, can be any of the urothelial cancer-associated genes, and panels thereof, described herein. The neoadjuvant treatment can be any of the neoadjuvant treatments described herein. The presence of the one or more mutations in the one or more urothelial cancer-associated genes can be detected by any of the procedures described herein. The methods may further comprise comparing the one or more mutations in the urothelial cancer-associated genes in the urine sample to germline mutations to filter out single nucleotide polymorphisms (SNPs), sequencing errors, and rare variants.

The present disclosure also provides methods for enhanced clinical staging and grading in UTUC. For example, the methods described herein can be used to detect high grade/high stage disease that might otherwise not be detected by a conventional ureteroscopic biopsy, which has high non-diagnostic rates and often understages disease. The methods described herein can be used for subjects undergoing an initial examination or any subsequent examination to initially establish high grade/high stage disease or to monitor the development of high grade/high stage disease. The methods described herein would be a significant advance in diagnostic urology. In some embodiments, methods of detecting a high grade and/or high stage UTUC disease are provided. In some embodiments, a urine sample obtained from the subject is analyzed for the presence of one or more mutations in one or more urothelial cancer-associated genes. If no mutations in urothelial cancer-associated genes which are associated with higher grade or stage (e.g., TP53, RB1, HRAS, KRAS, CDKN2A, CDKN1A, ATM, ERBB2, and/or ERBB3) are detected in the sample of urine, then the subject is diagnosed as not having a high grade and/or high stage UTUC disease. If mutations in one or more urothelial cancer-associated genes which are associated with higher grade or stage are detected in the sample of urine, then the subject is diagnosed as having a high grade and/or high stage UTUC disease.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Materials and Methods

### Gene Panel:

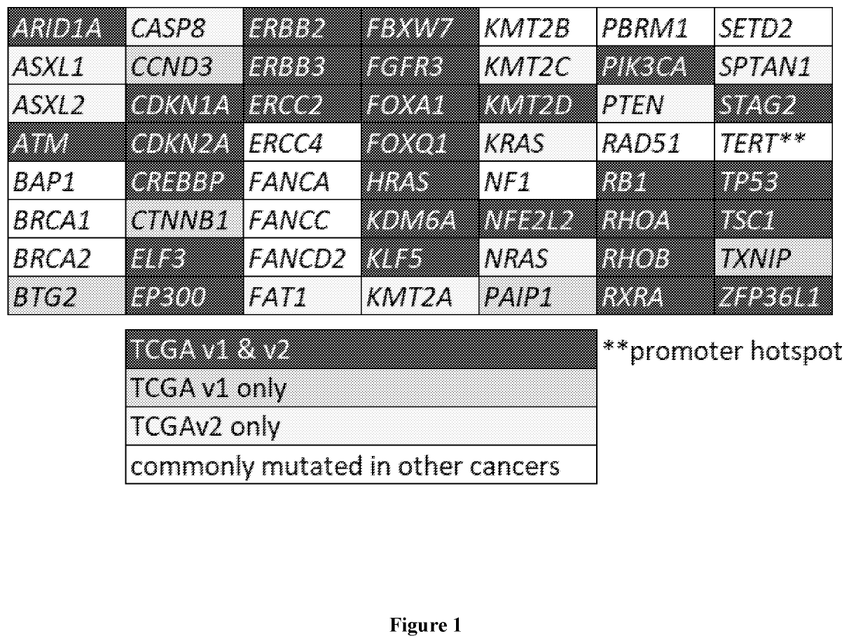

A panel of 56 MIBC genes (FIG. 1) was selected for targeted next generation sequencing (NGS). The panel covered exons of 43 TCGA-nominated driver genes (Cancer Genome Atlas Research N., Nature, 2014, 507, 315-22; see also, world wide web at “doi.org/10.1016/j.ce11.2017.09.007”), exons of an additional 12 genes identified as significantly mutated across all cancers (Lawrence et al., Nature, 2014, 505, 495-501) which are also common in MIBC, and TERT promoter hotspots (Huang et al., Science, 2013, 339, 957-9; and Horn et al., Science, 2013, 339, 959-61). UTUC driver genes have not been thoroughly described, but commonly mutated genes in UTUC have been described (Moss et al., Eur. Urol., 2017, 72, 641-9; and Sfakianos et al., Eur. Urol., 2015, 68, 970-7), and there is significant overlap between the MIBC and UTUC gene sets (at least 18 of the 24 genes described by Moss et al., Eur. Urol., 2017, 72, 641-9). Mutation of at least one gene in the panel occurs in 96-99% of bladder tumors across several studies (Cancer Genome Atlas Research N., Nature, 2014, 507, 315-22; Kim et al., Eur. Urol., 2015, 67, 198-201; and Van Allen et al., Cancer Discov., 2014, 4, 1140-53), at a median of 4 variants per tumor in the TCGA BLCA dataset.

### Samples:

Urine from 24 patients treated with methotrexate/vincristine/doxorubicin/cisplatin (MVAC) on a trial completed at Fox Chase Cancer Center was used. Four samples were not evaluable for molecular response due to either lack of a pre- or post-MVAC urine sample or QC failure. uDNA was isolated from urine supernatant using either the Norgen Urinary DNA Slurry Kit (part number 48800) or the Qiagen QlAamp Circulating Nucleic Acids Isolation Kit (part number 55114).

### Sequencing Method:

The HaloPlex^{HS} (Agilent) sequencing kit was used because it provides power to detect rare alleles by tagging each original DNA fragment with a single molecule barcode. Amplified barcodes can be used to make consensus sequences from duplicated amplicons to reduce false calls created by Taq error, and can also be used to count unique original fragments for a true depth-of-coverage estimate (Kinde et al., Proc. Natl. Acad. Sci. U.S.A., 2011, 108, 9530-5; and Smith et al., Genome Biol., 2014, 15, 420). This makes variant detection highly accurate even at low variant allele fraction (VAF). Indexed and barcoded HaloPlex^{HS} sequencing libraries were created from 50-200 ng of double-stranded uDNA as measured by Qubit. Libraries from each pre-/post-MVAC urine sample from these patients were pooled and deep sequenced. SureCall (Agilent) was used to call variants under standard conditions (at least 3 variant reads, at least 30 reads per site, Phred>30, mapping quality>30, strand bias<2:1). These conditions allow for some “noise” in sequencing reads without “overcalling” variants. Libraries were sequenced to a target sequencing depth of 1000x.

### Example 2

### Mutation Detection in Urine is Feasible and Accurate

WES and analysis was performed on the tumors of 15 MIBC patients, identifying 76 total mutations in the 56-gene panel. These variants were used as “benchmark” to characterize the sensitivity of the test for identifying tissue mutations (M_{T}) in the urine (M_{U}). Forty-six of the 76 M_{T} (61%) were identified in urine. Six of 14 M_{T} that were deemed subclonal (mutation allele fraction <0.8) were detected in uDNA. A majority of the M_{T} that were not identified as M_{U} were from four samples where no M_{T} were identified in uDNA. One these four patients likely achieved pCR prior to MVAC. The TURBT operative report described resection of a solitary tumor and post resection biopsy of the tumor bed which was pathologically benign. A second pre-MVAC urine sample was available from this patient and confirmed that no pre-MVAC M_{U} were present. pCR in the absence of NAC is a well-described phenomenon in patients undergoing RC in the absence of NAC (Grossman et al., N. Engl. J. Med., 2003, 349, 859-66; Canter et al., BJU Int., 2011, 107, 58-62; and Mak et al., J. Clin. Oncol., 2014, 32, 3801-9), and may have occurred for this patient, resulting in variant non-detection in pre-MVAC uDNA. In two of the other three benchmark patients without detectable pre-MVAC MU, M_{T} became detectable in urine after MVAC, suggesting that chemotherapy may augment shedding of tumor DNA to the urine.

In all, M_{T} called by whole exome sequencing (WES) were also called as pre-MVAC M_{U} in 11 of 15 cases. Overall, this data proves feasibility of M_{U} identification and engenders confidence that matching mutations can be found in tissue and urine.

### Example 3

### uDNA NGS is Highly Prognostic of pCR in RC and RNU Specimens

Pre-MVAC variants in the uDNA were identified using SureCall. These variants were compared against germline to filter SNPs, sequencing errors, and rare variants that may be associated with clonal hematopoiesis of indeterminate potential (Jaiswal et al., N. Engl. J. Med., 2014, 371, 2488-98). To determine if mutations persisted or cleared after NAC, post-MVAC sequencing libraries were searched using the genomic coordinates of the pre-MVAC mutations. Under the same calling conditions used for the previous analysis, mutation clearance or persistence status in the post-MVAC urine sample was ascribed at the mutation level. Subject-level mutation clearance was assigned only if all mutations demonstrated mutation clearance. Persistence of even one pre-MVAC mutation in the post-MVAC sample assigns subjects to mutation persistence status.

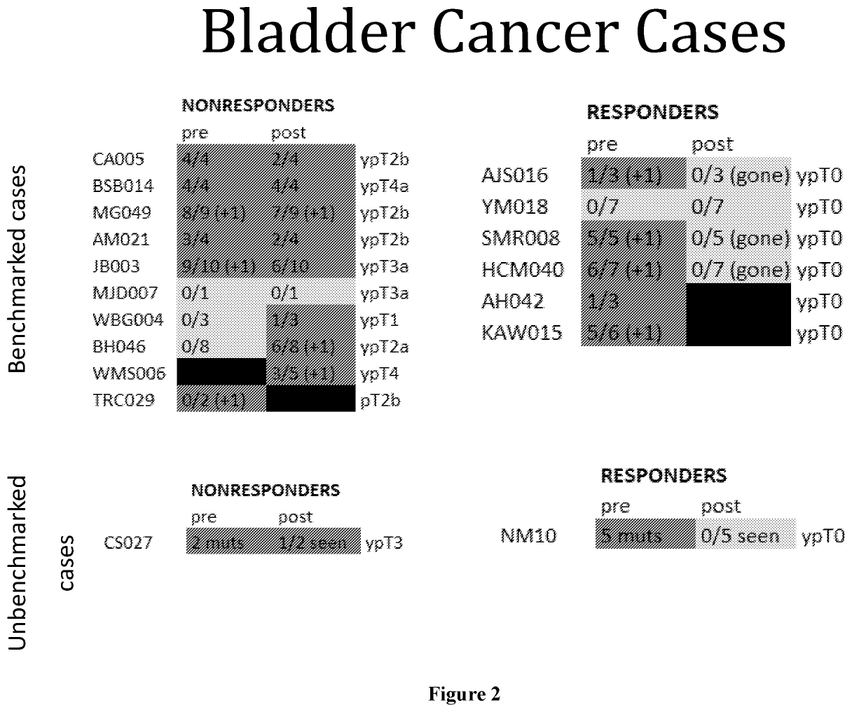

Referring to FIG. 2 (Upper Panel), benchmark cases were analyzed first to show that mutations in tumor tissue (M_{T}) are detectable in uDNA. The initial correlative analysis was limited to the 8 cases where pre-ddMVAC M_{U} were present and a post-MVAC uDNA sample was present. Mutation persistence was observed in all cases with residual disease and in no cases were pCR was achieved. When correlating pathologic residual disease with presence of mutation in only the presurgical urine specimen however, 8/9 patients with residual disease were identified as having persistent mutations present, whereas 4/4 patients achieving complete response have no detectable uDNA mutations. Additional unbenchmarked MIBC cases (Lower Panel) were analyzed and showed similar biomarker accuracy.

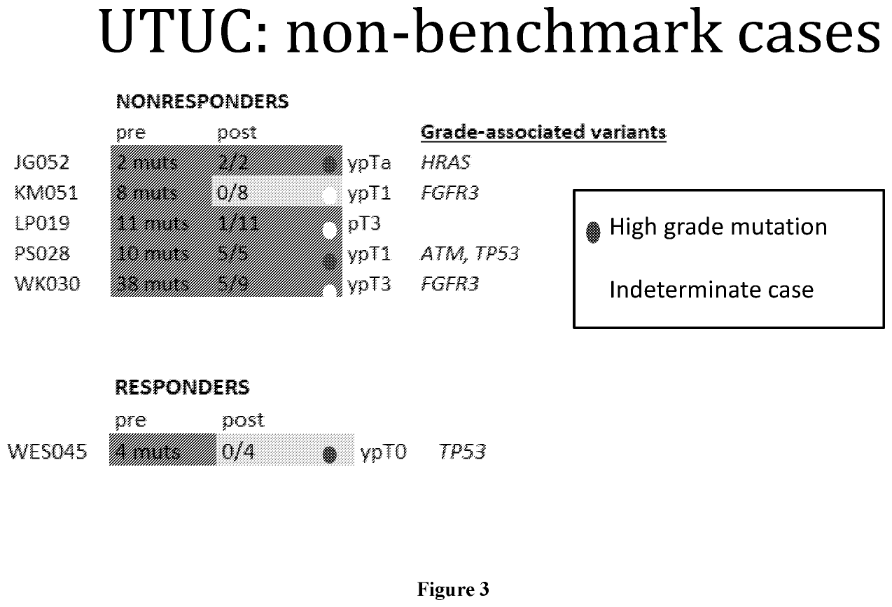

Referring to FIG. 3, UTUC cases were analyzed using the same approach. Pre-ddMVAC M_{U} were identified in all cases and persisted in 4 of 5 nonresponders but cleared in the complete responder.

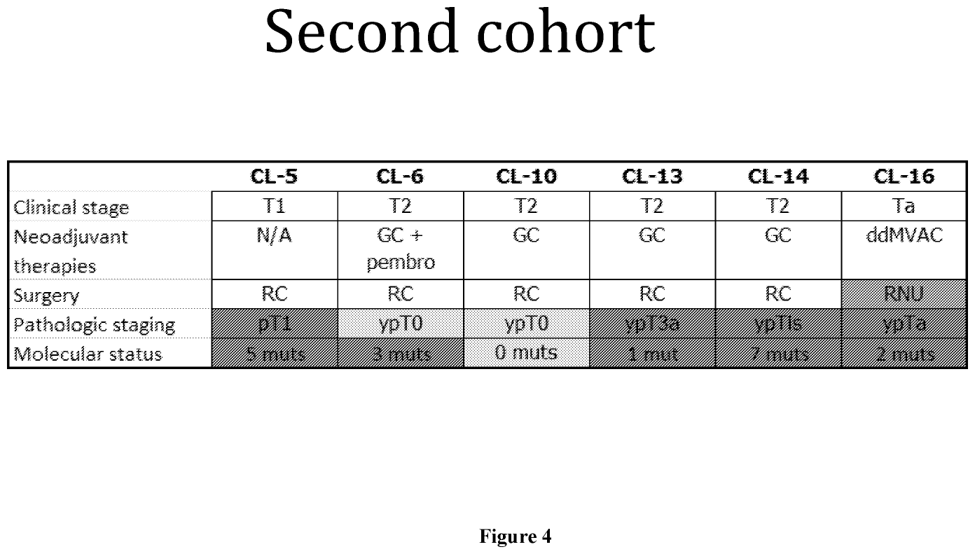

Referring to FIG. 4, an additional cohort of 6 patients who were treated at another institution using a variety of neoadjuvant therapies or no neoadjuvant therapy was also studied. Patients received no neoadjuvant chemotherapy (n=1), gemcitabine/cisplatin (n=3), gemcitabine/cisplatin/pembrolizumab (n=1) or ddMVAC (n=1). Only a pre-surgical urine sample was analyzed. Five of these patients underwent RC, and one underwent RNU. Persistent disease was detected in 4 patients at the time of organ removal, and all of these patients had persistent somatic mutations in their urine sample. Two of these patients had no detectable disease in their urine sample and one of these patients had absence of uDNA mutations while the other had detectable uDNA mutations.

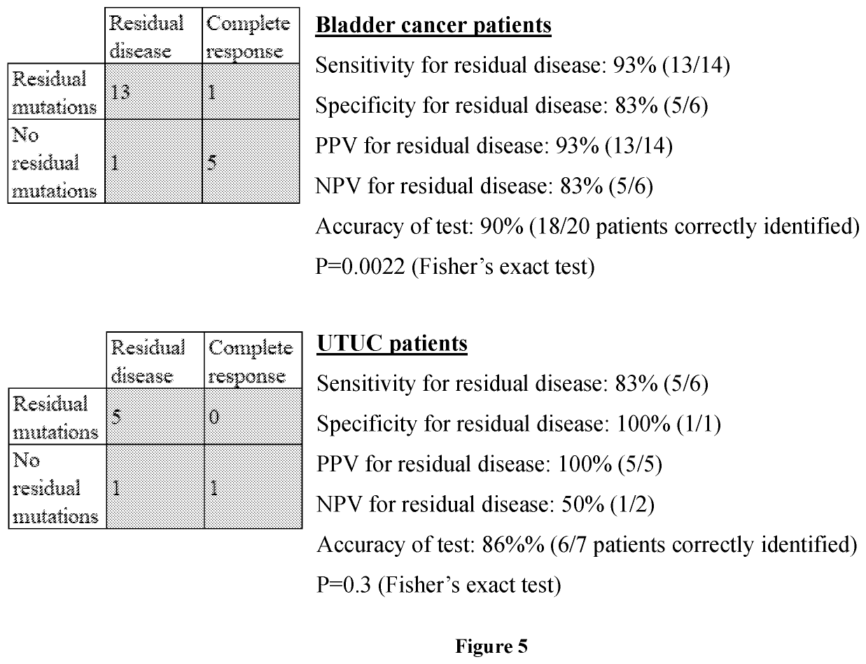

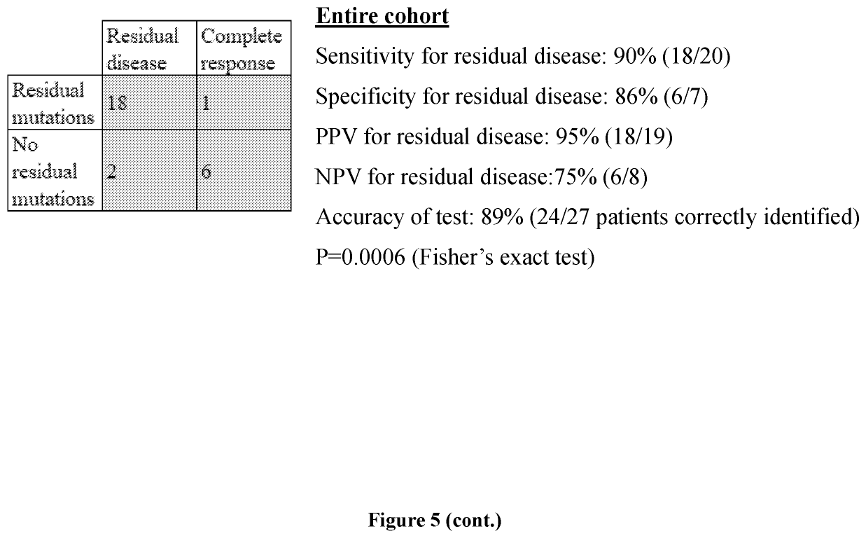

Referring to FIG. 5, when analyzing the entire cohort using patients who have a blood (germline) and pre-extirpation surgery urine sample, 27 patients were evaluable. The sensitivity of the test for detecting residual disease is 90% (18 of 20 patients with residual disease were found to have M_{U} prior to surgery). The PPV for residual disease in patients who test positive for M_{U} prior to surgery is 95% (18 of 19 patients who test positive for M_{U} prior to surgery have residual disease). The outcome of the urine test is highly correlated with responder status (p=0.0006, Fisher's exact test).

These data support the use of mutation persistence/clearance for the assignment of residual disease prior to RC or RNU and for the assignment of grade prior to radical nephroureterectomy.

### Example 4

### Mutations can be used to Assign Grade or Stage in UTUC

Additional analysis was performed to determine if mutations associated with grade or stage of disease. All aforementioned UTUC cases described in this application were high grade. Mutations in TP53, HRAS, ATM, CDKN1A, and deletion of CDKN2A are highly specific for high grade UTUC, whereas FGFR3 hotspot mutations are present in both high and low grade UTUC and are therefore indeterminant of cancer grade (Moss et al., Eur. Urol., 2017, 72, 641-9; Sfakianos et al., Eur. Urol., 2015, 68, 970-7). Mutations in genes associated with high grade disease were identified in 4 of 7 UTUC patients. This approach may, therefore, also be useful in assigning grade to disease using urine as a diagnostic medium. Although uDNA mutations were not confirmed in the tissue of UTUC patients, most patients had hotspot mutations in either the TERT promoter, HRAS, TP53, or FGFR3. Finding mutations at these previously noted hotspots which are common in urothelial tumors increases the likelihood that some or most of the other identified variants are true variants.

In summary: i) MIBC and UTUC patients shed adequate amounts of DNA into their urine, ii) uDNA sequencing is feasible, iii) mutation clearance status associates with pathologic response status, iv) stage and grade-specific mutations can be detected in the uDNA of UTUC patients, and v) the identified mutation profiles are consistent with published reports. Overall, the methods described herein may be used to prognosticate the pathological stage. In the aforementioned studies, patients who were clinically assessed to have no residual disease after chemotherapy have a 30-50% local recurrence rate when cystectomy is avoided, suggesting that clinical assessment is highly fallible. This urine test may significantly enhance clinical staging for use in identifying residual disease, or lack thereof, in anticipation of use in avoidance of radical cystectomy or radical nephroureterectomy. The methods may also be used to determine the pathological stage and grade in UTUC, a disease where current clinical assessment tools are unreliable and inaccurate. Therefore, the urine test could also be used to enhance clinical staging in order to more safely assign a treatment regimen.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of treating a subject having urothelial cancer comprising:
analyzing a urine sample obtained from the subject for the presence of one or more mutations in one or more urothelial cancer-associated genes; wherein:
i) if no mutations in urothelial cancer-associated genes are detected in the sample of urine, then refraining from removing the urinary bladder, ureter, or kidney, or any combination thereof, from the subject; and
ii) if mutations in one or more urothelial cancer-associated genes are detected in the sample of urine, then further removing the urinary bladder, ureter, or kidney, or any combination thereof, from the subject.

2. A method of treating a subject having urothelial cancer comprising:
administering neoadjuvant therapy to the subject; and
analyzing a urine sample obtained from the subject after receiving neoadjuvant treatment for the presence of one or more mutations in one or more urothelial cancer-associated genes; wherein:
i) if no mutations in urothelial cancer-associated genes are detected in the sample of urine, then refraining from removing the urinary bladder, ureter, or kidney, or any combination thereof, from the subject; and
ii) if mutations in one or more urothelial cancer-associated genes are detected in the sample of urine, then further removing the urinary bladder, ureter, or kidney, or any combination thereof, from the subject.

3. The method according to claim 2, further comprising:
analyzing a urine sample from the subject obtained prior to administering neoadjuvant therapy to the subject for the presence of one or more mutations in one or more urothelial cancer-associated genes; and
comparing the subject's urine mutation profile detected in the urine samples obtained prior to and after administration of neoadjuvant therapy; wherein:
i) if all of the mutations in one or more urothelial cancer-associated genes detected in the urine sample obtained prior to administering neoadjuvant therapy are not detected in the urine sample obtained after administering neoadjuvant therapy, then refraining from removing the urinary bladder, ureter, or kidney, or any combination thereof, from the subject; and
ii) if any of the mutations in one or more urothelial cancer-associated genes detected in the urine sample obtained prior to administering neoadjuvant therapy are also detected in the urine sample obtained after administering neoadjuvant therapy, then further removing the urinary bladder, ureter, or kidney, or any combination thereof, from the subject.

4. The method according to any one of claims 1 to 3, wherein the urothelial cancer is a muscle-invasive invasive urothelial cancer of the bladder, a non-muscle invasive urothelial cancer of the bladder, a ureteral cancer, or a renal pelvis cancer, or any combination thereof.

5. The method according to any one of claims 1 to 3, wherein the urothelial cancer is urothelial carcinoma, squamous cell carcinoma, small cell carcinoma, adenocarcinoma, micropapillary carcinoma, plasmacytoid carcinoma, or sarcomatoid differentiation, or any combination thereof.

6. The method according to any one of claims 1 to 5, wherein the urine sample is whole urine.

7. The method according to any one of claims 1 to 5, wherein the urine sample is urine sediment or urine supernatant.

8. The method according to any one of claims 1 to 7, wherein urinary DNA (uDNA) in the urine sample is analyzed for the presence of one or more mutations in one or more urothelial cancer-associated genes.

9. The method according to any one of claims 1 to 8, wherein at least 10 urothelial cancer-associated genes are analyzed for the presence of one or more somatic mutations.

10. The method according to claim 9, wherein the urothelial cancer-associated mutations are in TCGA-nominated urothelial cancer driver genes, TERT promoter, or genes identified as significantly mutated across all cancers which are also common in urothelial cancers.

11. The method according to claim 10, wherein the TCGA-nominated urothelial cancer driver genes are selected from the group consisting of ARID1A, ASXL1, ASXL2, ATM, BTG2, CASP8, CCND3, CDKN1A, CDKN2A, CREBBP, CTNNB1, ELF3, EP300, ERBB2, ERBB3, ERCC2, FAT1 , FBXW7, FGFR3, FOXA1, FOXQ1, HRAS, KDM6A, KLF5, KMT2A, KMT2C, KMT2D, KRAS, NFE2L2, NRAS, PIK3CA, PTEN, RB1, RHOA, RHOB, RXRA, SPTAN, STAG2, TP53, TSC1, TXNIP, and ZFP36L1.

12. The method according to claim 10, wherein the TCGA-nominated urothelial cancer driver genes are selected from the group consisting of TP53, KDM6A, KMT2D, ARID1A, KMT2C, RB1, EP300, FGFR3, STAG2, and ATM.

13. The method according to claim 10, wherein the genes identified as significantly mutated across all cancers which are also common in urothelial cancers are selected from the group consisting of BAP1, BRCA1, BRCA2, ERCC4, FANCA, FANCC, FANCD2, KMT2B, NF1, PAIP1, PBRM1, RAD51, and SETD2.

14. The method according to any one of claims 2 to 13, wherein the neoadjuvant treatment comprises administration of methotrexate/vincristine/doxorubicin/cisplatin (MVAC), gemcitabine/cisplatin, nivolumab, pembrolizumab, atezolizumab, durvalab, ipilumumab, avelumab, Bacillus-Calmette Guerin, doxorubicin, cisplatin, gemcitabine, docetaxel, thiotepa, or valrubicin, or any combination thereof.

15. The method according to any one of claims 1 to 14, wherein the presence of the one or more mutations in the one or more urothelial cancer-associated genes is detected by nucleic acid sequencing or in-situ hybridization.

16. The method according to any one of claims 1 to 15, further comprising comparing the one or more mutations in the urothelial cancer-associated genes in the urine sample to germline mutations to filter out single nucleotide polymorphisms (SNPs), sequencing errors, and rare variants.

17. The method according to any one of claims 1 to 16, further comprising treating the subject with an organ preservation regimen if no mutations in urothelial cancer-associated genes are detected in the sample of urine.

18. The method according to claim 17, wherein the organ preservation regimen comprises avoidance of radical cystectomy with or without urinary diversion, avoidance of radical nephroureterectomy, avoidance of distal ureterectomy and neocystostomy, or avoidance of irradiation of the bladder, renal pelvis, or ureter.

19. A method of monitoring the efficacy of neoadjuvant treatment in a subject having clinically localized urothelial cancer comprising:
analyzing a urine sample obtained from the subject prior to neoadjuvant treatment for the presence of one or more mutations in one or more urothelial cancer-associated genes;
administering neoadjuvant therapy to the subject;
analyzing a urine sample obtained from the subject after neoadjuvant treatment for the presence of one or more mutations in one or more urothelial cancer-associated genes; and
comparing the subject's somatic mutations detected in the urine samples obtained prior to and after administration of neoadjuvant therapy; wherein:
i) if all the subject's somatic mutations in the one or more urothelial cancer-associated genes detected in the urine sample obtained prior to administering neoadjuvant therapy are not detected in the urine sample obtained after administering neoadjuvant therapy, then the neoadjuvant treatment is deemed successful, and the subject is not further diagnosed as requiring removal of the urinary bladder, ureter, or kidney, or any combination thereof, from the subject; and
ii) if any of the subject's somatic mutations in the one or more urothelial cancer-associated genes detected in the urine sample obtained prior to administering neoadjuvant therapy are also detected in the urine sample obtained after administering neoadjuvant therapy, then the neoadjuvant treatment is deemed insufficient, and the subject is further diagnosed as requiring removal of the urinary bladder, ureter, or kidney, or any combination thereof, from the subject.

20. The method according to claim 19, wherein the urothelial cancer is a muscle-invasive invasive urothelial cancer of the bladder, a non-muscle invasive urothelial cancer of the bladder, a ureter cancer, or a renal pelvis cancer, or any combination thereof.

21. The method according to claim 19, wherein the urothelial cancer is urothelial carcinoma, squamous cell carcinoma, small cell carcinoma, adenocarcinoma, micropapillary carcinoma, plasmacytoid carcinoma, or sarcomatoid differentiation, or any combination thereof.

22. The method according to any one of claims 19 to 21, wherein the urine samples are whole urine.

23. The method according to any one of claims 19 to 21, wherein the urine samples are urine sediment or urine supernatant.

24. The method according to any one of claims 19 to 23, wherein urinary DNA (uDNA) in the urine samples is analyzed for the presence of one or more mutations in one or more urothelial cancer-associated genes.

25. The method according to any one of claims 19 to 24, wherein at least 10 urothelial cancer-associated genes are analyzed for the presence of one or more somatic mutations.

26. The method according to claim 25, wherein the urothelial cancer-associated mutations are in TCGA-nominated urothelial cancer driver genes, TERT promoter, or genes identified as significantly mutated across all cancers which are also common in urothelial cancers.

27. The method according to claim 26, wherein the TCGA-nominated urothelial cancer driver genes are selected from the group consisting of ARID1A, ASXL1, ASXL2, ATM, BTG2, CASP8, CCND3, CDKN1A, CDKN2A, CREBBP, CTNNB1, ELF3, EP300, ERBB2, ERBB3, ERCC2, FAT1 , FBXW7, FGFR3, FOXA1, FOXQ1, HRAS, KDM6A, KLF5, KMT2A, KMT2C, KMT2D, KRAS, NFE2L2, NRAS, PIK3CA, PTEN, RB1, RHOA, RHOB, RXRA, SPTAN, STAG2, TP53, TSC1, TXNIP, and ZFP36L1.

28. The method according to claim 26, wherein the TCGA-nominated urothelial cancer driver genes are selected from the group consisting of TP53, KDM6A, KMT2D, ARID1A, KMT2C, RB1, EP300, FGFR3, STAG2, and ATM.

29. The method according to claim 26, wherein the genes identified as significantly mutated across all cancers which are also common in urothelial cancers are selected from the group consisting of BAP1, BRCA1, BRCA2, ERCC4, FANCA, FANCC, FANCD2, KMT2B, NF1, PAIP1, PBRM1, RAD51, and SETD2.

30. The method according to any one of claims 19 to 29, wherein the neoadjuvant treatment comprises administration of methotrexate/vincristine/doxorubicin/cisplatin (MVAC), gemcitabine/cisplatin, nivolumab, pembrolizumab, atezolizumab, durvalab, ipilumumab, avelumab, Bacillus-Calmette Guerin, doxorubicin, cisplatin, gemcitabine, docetaxel, thiotepa, or valrubicin, or any combination thereof.

31. The method according to any one of claims 19 to 30, wherein the presence of the one or more mutations in the one or more urothelial cancer-associated genes is detected by nucleic acid sequencing or in-situ hybridization.

32. The method according to any one of claims 19 to 31, further comprising comparing the one or more mutations in the urothelial cancer-associated genes in the urine sample to germline mutations to filter out single nucleotide polymorphisms (SNPs), sequencing errors, and rare variants.

33. The method according to any one of claims 19 to 32, further comprising treating the subject with an organ preservation regimen if no mutations in urothelial cancer-associated genes are detected in the sample of urine obtained after administration of neoadjuvant therapy.

34. The method according to claim 33, wherein the organ preservation regimen comprises avoidance of radical cystectomy with or without urinary diversion, avoidance of radical nephroureterectomy, avoidance of distal ureterectomy and neocystostomy, or avoidance of irradiation of the bladder, renal pelvis, or ureter.

35. A method of analyzing a subject having urothelial cancer comprising:
analyzing a urine sample obtained from the subject prior to neoadjuvant treatment for the presence of one or more mutations in one or more urothelial cancer-associated genes;
administering neoadjuvant therapy to the subject;
analyzing a urine sample obtained from the subject after neoadjuvant treatment for the presence of one or more mutations in one or more urothelial cancer-associated genes; and
comparing the subject's urine mutation profile detected in the urine samples obtained prior to and after administration of neoadjuvant therapy.

36. A method of treating a subject having urothelial cancer comprising:
requesting a test for the subject, wherein the test analyzes urine samples obtained from the subject prior to and after neoadjuvant treatment for one or more mutations in one or more urothelial cancer-associated genes;
examining the result of the test, wherein the result indicates whether or not all of the mutations in the urothelial cancer-associated genes identified in the urine sample obtained prior to neoadjuvant treatment are absent from the urine sample obtained after neoadjuvant treatment; and
i) if all the mutations in the urothelial cancer-associated genes identified in the urine sample obtained prior to neoadjuvant treatment are absent from the urine sample obtained after neoadjuvant treatment, then refraining from removing the urinary bladder, ureter, or kidney, or any combination thereof, from the subject; or
ii) if all the mutations in the urothelial cancer-associated genes identified in the urine sample obtained prior to neoadjuvant treatment are not absent from the urine sample obtained after neoadjuvant treatment, then further removing the urinary bladder, ureter, or kidney, or any combination thereof, from the subject.

37. A method of treating a subject having urothelial cancer comprising:
examining the result of a test for the subject, wherein the test analyzes urine samples obtained from the subject prior to and after neoadjuvant treatment for one or more mutations in one or more urothelial cancer-associated genes, and wherein the result indicates whether or not all of the mutations in the urothelial cancer-associated genes identified in the urine sample obtained prior to neoadjuvant treatment are absent from the urine sample obtained after neoadjuvant treatment; and
i) if all the mutations in the urothelial cancer-associated genes identified in the urine sample obtained prior to neoadjuvant treatment are absent from the urine sample obtained after neoadjuvant treatment, then refraining from removing the urinary bladder, ureter, or kidney, or any combination thereof, from the subject; or
ii) if all the mutations in the urothelial cancer-associated genes identified in the urine sample obtained prior to neoadjuvant treatment are not absent from the urine sample obtained after neoadjuvant treatment, then further removing the urinary bladder, ureter, or kidney, or any combination thereof, from the subject.
